(19) United States
(12) Patent Application Publication
(10) Pub. No.: US 2025/0223473 A1
(43) Pub. Date: Jul. 10, 2025

(54) LONG TERM WEARABLE MEDICAL PRESSURE SENSITIVE ADHESIVE ARTICLES

(71) Applicant: Solventum Intellectual Properties Company, Maplewood, MN (US)

(72) Inventors: Yasuhiro Kinoshita, Tokyo (JP); James P. DiZio, St. Paul, MN (US); Bret W. Ludwig, Oakdale, MN (US); Audrey A. Sherman, Woodbury, MN (US)

(21) Appl. No.: 18/852,592

(22) PCT Filed: Mar. 22, 2023
(86) PCT No.: PCT/IB2023/052830

§ 371 Date: Sep. 30, 2024

Related U.S. Application Data
(60) Provisional application No. 63/332,063, filed on Apr. 18, 2022.

(51) Int. Cl. C09J 133/08; A61L 15/58; C09J 7/38

(52) U.S. Cl. CPC C09J 133/08; A61L 15/585; C09J 7/385; C09J 2301/302; C09J 2301/414; C09J 2433/00

(57) ABSTRACT

Pressure sensitive adhesive articles include a substrate with a first major surface and a second major surface, and a pressure sensitive adhesive layer disposed on at least a portion of the first major surface of the substrate. The pressure sensitive adhesive layer is an E-beam cured composition of a (meth)acrylate-based polymer that is free from acidic-functional or amide-functional groups and at least one tackifier that is a hydrogenated hydrocarbon resin. The pressure sensitive adhesive layer has a static shear of at least 600 minutes to protein leather.

### SUMMARY

Disclosed herein are pressure sensitive adhesive articles and medical constructions prepared using the pressure sensitive adhesive articles. In some embodiments, the pressure sensitive adhesive article comprises a substrate with a first major surface and a second major surface, and a pressure sensitive adhesive layer disposed on at least a portion of the first major surface of the substrate. The pressure sensitive adhesive layer comprises an E-beam cured composition comprising a (meth)acrylate-based polymer that is free from acidic-functional or amide-functional groups, at least one tackifier comprising a hydrogenated hydrocarbon resin. The pressure sensitive adhesive layer has a static shear of at least 600 minutes to PROTEIN LEATHER.

Also disclosed are medical constructions. In some embodiments, the medical construction comprises a surface comprising mammalian skin, and an adhesive article adhesively attached to the surface. The adhesive article has been described above.

### BRIEF DESCRIPTION OF THE DRAWINGS

The present application may be more completely understood in consideration of the following detailed description of various embodiments of the disclosure in connection with the accompanying drawings.

FIG. 1 is a graph of Static Shear on Sebum coated PROTEIN LEATHER for Examples E21, E22, E24, and E25

### DETAILED DESCRIPTION

The use of adhesive products in the medical industry has long been prevalent and is increasing. However, while adhesives and adhesive articles have shown themselves to be very useful for medical applications, there are also issues in the use of adhesives and adhesive articles. In particular, the desired properties for adhesives are often contradictory. For example, it is desirable that the adhesives have high adhesion to an array of surfaces, including human skin, and yet the adhesive also is desirably removable without damaging the skin. Additionally, medical articles are being worn for longer periods of time, needing to remain adhered and yet need to be removable without damaging the skin or leaving residue.

Medical adhesive-related skin injury (MARSI) has a significant negative impact on patient safety. Skin injury related to medical adhesive usage is a prevalent but under recognized complication that occurs across all care settings and among all age groups. In addition, treating skin damage is costly in terms of service provision, time, and additional treatments and supplies.

Skin Injury occurs when the superficial layers of the skin are removed along with the medical adhesive product, which not only affects skin integrity but can cause pain and the risk of infection, increase wound size, and delay healing, all of which reduce patients' quality of life

The pathophysiology of MARSI is only partially understood. Skin injury results when the skin to adhesive attachment is stronger than skin cell to skin cell attachment. When adhesive strength exceeds the strength of skin cell to skin cell interactions, cohesive failure occurs within the skin cell layer.

Typical medical adhesive articles include an adhesive layer and a substrate layer, where the substrate layer may for example be a tape backing. Other medical adhesive articles have other substrate layers and may include multiples layers, devices, and the like. The intrinsic characteristics of all components of an adhesive article must then be taken into account to address these factors that may lead to MARSI. Properties of the adhesive to be considered include cohesiveness over time and the corresponding adhesion strength; properties of the tape/backing/dressing to be considered include breathability, stretch, conformability, flexibility, and strength.

The widespread use of adhesives in medical applications has led to the development of adhesives and adhesive articles that are gentle to the skin. Some of these adhesives are pressure sensitive adhesives. The application of pressure sensitive adhesives, including (meth)acrylate-based and silicone-based pressure sensitive adhesives, for adhering to skin is known in the art and many examples are commercially available.

Among the classes of adhesive materials that have found widespread use as pressure sensitive adhesives are (meth)acrylate-based pressure sensitive adhesives. These materials have many desirable features such as frequently being inherently tacky and thus not requiring the use of added tackifying agents, they are typically formed by free radical polymerization to a high conversion, meaning that little or no un-polymerized monomer is left in the formed pressure sensitive adhesive, and a wide range of monomers can be used to form (meth)acrylate-based copolymers to tailor the desired properties of the pressure sensitive adhesive. Frequently the (meth)acrylate-based pressure sensitive adhesive is prepared from a reaction mixture that contains monomers with polar groups such as acidic and basic groups. Acidic and basic monomers are often classified in the adhesive art as reinforcing monomers, as these monomers tend to increase the cohesive strength of (meth)acrylate-based pressure sensitive adhesives. Therefore, preparing (meth)acrylate-based pressure sensitive adhesives that retain the requisite cohesive strength to be useful in medical applications without including acidic or basic reinforcing monomers is a challenge.

The term “adhesive” as used herein refers to polymeric compositions useful to adhere together two adherends. Examples of adhesives are pressure sensitive adhesives.

Pressure sensitive adhesive compositions are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as pressure sensitive adhesives are polymers designed and formulated to exhibit the requisite viscoelastic properties resulting in a desired balance of tack, peel adhesion, and shear holding power. Obtaining the proper balance of properties is not a simple process.

The term “(meth)acrylate-based” refers to polymers that contain at least (meth)acrylate monomers and may also contain additional co-polymerizable monomers.

The term “(meth)acrylate” refers to monomeric acrylic or methacrylic esters of alcohols. Acrylate and methacrylate monomers or oligomers are referred to collectively herein as “(meth)acrylates”.

The term “PROTEIN LEATHER” is used herein, according to its commonly understood meaning. Protein leather, also known as Pleather, is composed of protein powder along with resin to form pliable sheets. These sheets are a look-alike to leather in appearance as well as durability.

The term “hydrocarbon-based” is used herein to describe tackifying resins and plasticizers refers to materials that are hydrocarbons, meaning they contain carbon and hydrogen atoms, and are essentially free of functional groups.

The term “hydrogenated” is used herein to describe materials such as tackifying resins that are either fully hydrogenated, meaning that the material or resin is substantially free from unsaturated groups, or partially hydrogenated meaning that a substantial amount of the unsaturation in the material or resin has been hydrogenated, typically 70% or more.

The terms “room temperature” and “ambient temperature” are used interchangeably to mean temperatures in the range of 20° C. to 25° C.

The term “adjacent” as used herein when referring to two layers means that the two layers are in proximity with one another with no intervening open space between them. They may be in direct contact with one another (e.g. laminated together) or there may be intervening layers.

The terms “polymer” and “macromolecule” are used herein consistent with their common usage in chemistry. Polymers and macromolecules are composed of many repeated subunits. As used herein, the term “macromolecule” is used to describe a group attached to a monomer that has multiple repeating units. The term “polymer” is used to describe the resultant material formed from a polymerization reaction.

The term “alkyl” refers to a monovalent group that is a radical of an alkane, which is a saturated hydrocarbon. The alkyl can be linear, branched, cyclic, or combinations thereof and typically has 1 to 20 carbon atoms. In some embodiments, the alkyl group contains 1 to 18, 1 to 12, 1 to 10, 1 to 8, 1 to 6, or 1 to 4 carbon atoms. Examples of alkyl groups include, but are not limited to, methyl, ethyl, n-propyl, isopropyl, n-butyl, isobutyl, tert-butyl, n-pentyl, n-hexyl, cyclohexyl, n-heptyl, n-octyl, and ethylhexyl.

The term “aryl” refers to a monovalent group that is aromatic and carbocyclic. The aryl can have one to five rings that are connected to or fused to the aromatic ring. The other ring structures can be aromatic, non-aromatic, or combinations thereof. Examples of aryl groups include, but are not limited to, phenyl, biphenyl, terphenyl, anthryl, naphthyl, acenaphthyl, anthraquinonyl, phenanthryl, anthracenyl, pyrenyl, perylenyl, and fluorenyl. The term “heteroalkyl” refers to a monovalent group of an alkyl with one or more interstitial heteroatoms. The heteroatoms are —O— or —NR— where R is an H atom or alkyl group.

The terms “free radically polymerizable” and “ethylenically unsaturated” are used interchangeably and refer to a reactive group which contains a carbon-carbon double bond which is able to be polymerized via a free radical polymerization mechanism.

Disclosed herein are pressure sensitive adhesive articles. The adhesive articles comprise a substrate with a pressure sensitive adhesive layer disposed on at least a portion of a surface of the substrate. The substrates and pressure sensitive adhesive layers are described in detail below. The adhesive articles of the current disclosure have a wide range of desirable properties. Among the desirable properties are wearability for long periods of time without causing skin damage. One of the measures for the modeling the desirable features for such an adhesive article is the measurement of static shear. Static shear measurement, especially on a surface that mimics mammalian, in particular human, skin provides a mimic of the long term wearability of an adhesive article. In this disclosure, PROTEIN LEATHER is used as a particularly suitable test surface. PROTEIN LEATHER refers to artificial leather (sometimes called pleather) that is composed of protein powder along with resin to form pliable sheets. These sheets are a look-alike to leather in appearance as well as durability. The use of PROTEIN LEATHER in testing of samples is explained in detail in the Examples section. One particularly suitable PROTEIN LEATHER is PROTEIN LEATHER PBZ13001 KAKI form IDEATEX Japan Co.

As mentioned above, it is desirable that the adhesive article be removable from mammalian skin without causing skin damage. In some embodiments, the adhesive article is removable after 5 days. In many embodiments, the adhesive article is removable after longer periods of time such as 7 days, 14 days, 21 days, 30 days or longer. Typically, skin damage is determined by a physical inspection of the site to which the adhesive article was attached.

In some embodiments the pressure sensitive adhesive article comprises a substrate with a first major surface and a second major surface; and a pressure sensitive adhesive layer disposed on at least a portion of the first major surface of the substrate. The pressure sensitive adhesive layer comprises an E-beam cured composition comprising a (meth)acrylate-based polymer that is free from acidic-functional or amide-functional groups, and at least one tackifier comprising a hydrogenated hydrocarbon resin. In some embodiments, the pressure sensitive adhesive article further comprises at least one hydrocarbon-based plasticizer. The pressure sensitive adhesive layer has a static shear of at least 600 minutes to PROTEIN LEATHER. In some embodiments, the adhesive article has a static shear of at least 1,000 minutes, at least 2,000 minutes, or even at least 2,880 minutes. The method used to measure the static shear on PROTEIN LEATHER is described in the Examples section below.

The adhesive articles of this disclosure comprise a substrate. A wide variety of substrates are suitable for the articles of this disclosure. In many embodiments, the substrate comprises a substrate suitable for use in a medical article. Examples of suitable substrates include a polymeric film, a fabric, a non-woven, a foam, a paper, a mesh, an adhesive, or a release liner. In some embodiments, the substrate comprises a breathable conformable backing such as high moisture vapor permeable film backings. Examples of such backings, methods of making such films, and methods for testing their permeability are described, for example, in U.S. Pat. Nos. 3,645,835 and 4,595,001.

Generally, the substrate is conformable to anatomical surfaces. As such, when the article is applied to an anatomical surface, it conforms to the surface even when the surface is moved. Generally, the substrate is also conformable to animal anatomical joints. When the joint is flexed and then returned to its unflexed position, the substrate stretches to accommodate the flexion of the joint but is resilient enough to continue to conform to the joint when the joint is returned to its unflexed condition.

Examples of particularly suitable film backings can be found in U.S. Pat. Nos. 5,088,483 and 5,160,315, and include elastomeric polyurethane, polyester, or polyether block amide films. These films have a combination of desirable properties including resiliency, high moisture vapor permeability, and transparency.

The pressure sensitive adhesive articles of this disclosure also comprise a pressure sensitive adhesive layer disposed on the substrate. The pressure sensitive adhesive layer comprises primarily a (meth)acrylate-based polymer. By this it is meant that the (meth)acrylate-based polymer content of the adhesive layer is greater than 50% by weight. In some embodiments, the amount of the (meth)acrylate-based polymer present in the pressure sensitive adhesive layer is at least 70% by weight.

The (meth)acrylate-based polymer is prepared from a polymerized reaction mixture, where the reaction mixture comprises at least a first (meth)acrylate monomer, at least one co-polymerizable polar monomer that is free of acidic or amide functional groups, and at least one free radical initiator.

In some embodiments, the reaction mixture comprises at least 75 parts by weight of a first (meth)acrylate monomer of general formula I:

CH₂═CR¹—(CO)—OR² Formula I

where R¹ is hydrogen or a methyl group, and R² is an alkyl, heteroalkyl, or aryl group. A wide range of (meth)acrylate monomers are suitable. In some embodiments, the first (meth)acrylate monomer comprises an alkyl (meth)acrylate monomer with 4-12 carbon atoms. Examples monomers include, but are not limited to, those selected from the group consisting of the esters of acrylic acid or methacrylic acid with non-tertiary alkyl alcohols such as 1-butanol, 1-pentanol, 2-pentanol, 3-pentanol, 2-methyl-1-butanol, 1-methyl-1-butanol, 1-methyl-1-pentanol, 2-methyl-1-pentanol, 3-methyl-1-pentanol, 2-ethyl-1-butanol, 2-ethyl-1-hexanol, 3,5,5-trimethyl-1-hexanol, 3-heptanol, 2-octanol, 1-decanol, 1-dodecanol, and the like, and mixtures thereof. Such monomeric acrylic or methacrylic esters are known in the art and are commercially available. Particularly suitable are alkyl (meth)acrylate monomers with 8-12 carbon atoms such a 2-ethylhexyl acrylate and iso-octyl acrylate.

The reaction mixture used to form the (meth)acrylate-based polymer additionally contains at least one co-polymerizable polar monomer that is free of acidic or amide functional groups. Acidic or amide-functional (meth)acrylate monomers are typically copolymerized in (meth)acrylate polymers used to form pressure sensitive adhesives in order to increase the internal cohesive strength in the pressure sensitive adhesive. Pressure sensitive adhesives prepared from polymers that only contain alkyl (meth)acrylate monomers tend to be cohesively very weak.

The current reaction mixtures do not contain acid-functional or amide-functional (meth)acrylate monomers as the presence of these monomers if pressure sensitive adhesive polymers can cause skin damage issues when adhesive articles are worn for extended periods of time. Therefore, polar monomers that are not acid-functional or amide-functional monomers are used.

Representative examples of suitable polar monomers include, but are not limited to: 2-hydroxyethyl (meth)acrylate; 4-hydroxybutyl (meth)acrylate; N-vinylpyrrolidone (NVP); N-vinylcaprolactam (NVC); poly(alkoxyalkyl) (meth)acrylates including 2-(2-ethoxyethoxy)ethyl (meth)acrylate, 2-ethoxyethyl (meth)acrylate, 2-methoxyethoxyethyl (meth)acrylate, 2-methoxyethyl methacrylate, and polyethylene glycol mono(meth)acrylate; alkyl vinyl ethers, including vinyl methyl ether; and mixtures thereof. One particularly suitable polar monomer is NVP (N-vinyl pyrrolidone.

The amount of polar monomer present in the reaction mixture can vary. Typically, the polar monomer is present in an amount of at least 1% by weight and typically is not present in an amount greater than 30% by weight. More typically the polar monomer is present in an amount of from 2-20% by weight.

In addition to the monomers listed above, the reaction mixture may further comprise one or more additional co-polymerizable monomers. A wide range of co-polymerizable monomers are suitable. In some embodiments, the co-polymerizable monomer comprises a co-polymerizable photocrosslinker. Co-polymerizable photocrosslinkers are materials that contain a free radically polymerizable group to co-polymerize with the monomers described above. The co-polymerizable photocrosslinkers also contain a photosensitive group that upon exposure to the right wavelength of light, typically high intensity ultra-violet (UV) radiation, the photosensitive group forms free radicals which can form crosslinks in the polymer. If the (meth)acrylate-based polymer is formed by the use of a photoinitiator, the photocrosslinker is not activated by the same wavelengths of light as the photoinitiator. In this way, the co-polymerizable photocrosslinker is incorporated into the polymer, and is able to be thermally processed, as the crosslinker is thermally stable and remains intact until activated by the proper wavelength of light. This permits the co-polymerizable photocrosslinker from becoming activated until the polymer has been hot melt coated. The coated crosslinkable pressure sensitive adhesive layer is subjected to exposure to high intensity UV lamps to effect crosslinking. Examples of suitable UV lamps include medium pressure mercury lamps or a UV blacklight.

Suitable photocrosslinkers in the mono-ethylenically unsaturated aromatic ketone co-monomers that are free of ortho-aromatic hydroxyl groups such as those described in U.S. Pat. No. 4,737,559 (Kellen et al.). Specific examples include para-acryloxybenzophenone (ABP also sometimes referred to as AEBP), para-acrylyoxyethoxybenzophenone, para-N-(methylacryloxyethyl)-carbamoylethoxybenzophenone, para-acryloxyacetophenone, ortho-acrylamidoacetophenone, acrylated anthraquinones, and the like. Particularly suitable is ABP para-acryloxybenzophenone also called 4-acryloxybenzophenone.

Typically, such photocrosslinkers are used in amounts of about 0.05-0.50 phr. The term phr means Parts per Hundred Rubber, a measure used in the rubber industry to depict what amount of certain ingredients are needed, especially pre-vulcanization. In this case, the term phr refers to the parts by weight of photocrosslinker per 100 parts by weight of total monomers present in the reaction mixture. In some embodiments, the photocrosslinker is present in amounts of about 0.10 parts by weight of crosslinker per 100 parts by weight of total monomers present in the reaction mixture.

Because, as will be described in greater detail below, the pressure sensitive adhesives are subjected to crosslinking via exposure to electron beam radiation, the use of photocrosslinkers is not necessary, however such materials can aid in forming the crosslinked pressure sensitive adhesives by providing additional crosslinking.

The reaction mixture also comprises at least one free radical initiator. The initiator may be a thermal initiator or a photoinitiator. Thermal initiators are those that are activated to form free radicals upon exposure to an elevated temperature. Photoinitiators are those that are activated by light, typically ultraviolet (UV) light. Selection of the initiator depends upon a variety of factors, especially the composition of the reaction mixture. If a photocrosslinker is used, a photointitor is not desirable as exposure to light such as UV light can pre-maturely activate the photocrosslinker. In many embodiments a thermal initiator is used.

Many possible thermal free radical initiators are known in the art of vinyl monomer polymerization and may be used. Typical thermal free radical polymerization initiators which are useful herein are organic peroxides, organic hydroperoxides, and azo-group initiators which produce free radicals. Useful organic peroxides include but are not limited to compounds such as benzoyl peroxide, di-t-amyl peroxide, t-butyl peroxy benzoate, and di-cumyl peroxide. Useful organic hydroperoxides include but are not limited to compounds such as t-amyl hydroperoxide and t-butyl hydroperoxide. Useful azo-group initiators include but are not limited to the VAZO compounds manufactured by DuPont, such as VAZO 52 (2,2′-azobis(2,4-dimethylpentanenitrile)), VAZO 64 (2,2′-azobis(2-methylpropanenitrile)), VAZO 67 (2,2′-azobis(2-methylbutanenitrile)), and VAZO 88 (2,2′-azobis(cyclohexanecarbonitrile)). Additional commercially available thermal initiators include, for example, LUPERSOL 130 (2,5-dimethyl-2,5-Di-(t-butylperoxy) hexyne-3) available from Elf Atochem, Philadelphia, PA, and LUPEROX 101 (2,5-dimethyl-2,5-di-(tert-butylperoxoxy) hexane) available from Arkema Canada, Inc., Oakville. In US Patent Publication No. 2011/0300296, the polymerization process is described in detail and in some embodiments includes a mixture of initiators.

In some embodiments, the initiator is a photoinitiator. Examples of suitable free radical photoinitiators include DAROCURE 1173, DAROCURE 4265, IRGACURE 184, IRGACURE 651, IRGACURE 1173, IRGACURE 819, LUCIRIN TPO, LUCIRIN TPO-L, commercially available from BASF, Charlotte, NC. The photoinitiator DAROCURE 1173 is particularly suitable.

Generally, the initiator is used in amounts of 0.01 to 2 parts by weight, more typically 0.1 to 0.5, parts by weight relative to 100 parts by weight of total reactive components.

The pressure sensitive adhesive layer further comprises at least one tackifier comprising a hydrogenated hydrocarbon resin. The tackifier is a minor component in the pressure sensitive adhesive layer, meaning it comprises less than 50% by weight of the total components of the layer. Typically, the tackifier is present in an amount of from 5-40% by weight, or 10-30% by weight, or 15-25% by weight.

Conventional (meth)acrylate pressure sensitive adhesives use tackifier resins that include materials such as rosin esters and terpene phenolic resins that are not suitable in the current adhesive articles. Rosin esters and terpene phenolic resins are frequently used tackifying resins because they have high compatibility with (meth)acrylate polymers. The rosin esters and terpene phenolic resins are unsuitable tackifying resins for the current adhesive articles because they have the potential to cause skin sensitivity. This is especially true in the current articles that are crosslinked by exposure to an electron beam, because such exposure can cause decomposition of rosin esters and terpene phenolic resins to form skin irritating small molecules. Therefore, the current adhesive articles use hydrogenated hydrocarbon-resins. In some instances, the hydrogenated hydrocarbon resins are fully hydrogenated, in some instances the hydrogenated hydrocarbon are partially hydrogenated. Partially hydrogenated hydrocarbon resins may be suitable as long as it has sufficient compatibility with the remaining components of the pressure sensitive adhesive. While fully hydrogenated hydrocarbon resins are in many embodiments, preferred, partially hydrogenated hydrocarbon resins may be useful. The fully hydrogenated hydrocarbon tackifier resins are substantially free of unsaturated groups and also is free of polar groups. A wide range of hydrogenated hydrocarbon tackifier resins are suitable. Among the suitable resins are those available from Aquent Impex under the trade names ES300, ES320, ES340, ES380, ES600, and ES615 as well as a number of resins available from Arakawa Chemical. Examples of suitable resins form Arakawa Chemical include those under the ARKON trade name such as the ARKON M series (partially hydrogenated) such as ARKON M-100, ARKON M-115, ARKON M-135, and ARKON M-90, and the ARKON P series (fully hydrogenated) such as ARKON P-100, ARKON P-115, ARKON P-125, ARKON P-140, and ARKON P-90. One particularly suitable tackifier is ARKON P-100.

In some embodiments, the pressure sensitive adhesive articles of this disclosure further comprise at least one hydrocarbon-based plasticizer. Plasticizers are common additives used in many polymeric compositions such as pressure sensitive adhesives. Plasticizers are typically added to increase flexibility or workability of the polymer system. Plasticizers typically affect the viscosity, lower the glass transition temperature, and lower the elastic modulus of the polymer composition. Among the typical classes of plasticizers are phthalates and terephthalates. Plasticizers, like tackifiers, are chosen for their compatibility with the polymer composition. Among the typical plasticizers used with (meth)acrylate-based pressure sensitive adhesives are phthalates, terephthalates, benzoates, and epoxidized oils such as Epoxidized Soybean Oil (ESO).

The choice of suitable plasticizers in the current pressure sensitive adhesive layers is limited in a variety of ways. The chemical nature of the current pressure sensitive adhesive layers place limitations on what plasticizers are suitable, and the use of these pressure sensitive adhesive layers in medical articles also limits the choices of suitable plasticizers. Since the current pressure sensitive adhesive layers include hydrogenated hydrocarbon resins, many conventional plasticizers are unsuitable. Conventional plasticizers do not have a high compatibility with these hydrogenated hydrocarbon materials. Therefore, if plasticizers are used in the current pressure sensitive adhesive layers, they are typically hydrocarbon-based, since these plasticizers have a high compatibility with the hydrogenated hydrocarbon resins. In this way, the plasticizers help to compatibilize the (meth)acrylate-based polymer and the hydrogenated hydrocarbon tackifier resins in the pressure sensitive adhesive layer. In addition, as mentioned above, the plasticizer has to be biocompatible, meaning that the plasticizer does not cause adverse reactions when applied to skin. Examples of suitable plasticizers include IOP (iso-octyl palmitate), the polyester polyol PRIPLAST 3197 available from Croda, tea tree oil, and mineral oils.

As mentioned above, the pressure sensitive adhesive layer is a crosslinked pressure sensitive adhesive layer, where the crosslinking is carried out by exposure to electron beam (E-beam) radiation. Crosslinking is used to increase the cohesive strength of the pressure sensitive adhesive layer. E-beam curing is particularly desirable in the current articles since no initiator is necessary to carry out the crosslinking and therefore no initiator residues are left in the crosslinked pressure sensitive adhesive layer.

A variety of procedures for E-beam curing are well-known. The cure depends on the specific equipment used, and those skilled in the art can define a dose calibration model for the specific equipment, geometry, and line speed, as well as other well understood process parameters.

Commercially available electron beam generating equipment is readily available. For the examples described herein, the radiation processing was performed on a Model CB-300 electron beam generating apparatus (available from Energy Sciences, Inc. (Wilmington, MA). Generally, a support film (e.g., polyester terephthalate support film) runs through a chamber. In some embodiments, a sample of uncured material with a liner (e.g., a fluorosilicone release liner) on both sides (“closed face”) may be attached to the support film and conveyed at a fixed speed of about 6.1 meters/min (20 feet/min). In some embodiments, a sample of the uncured material may be applied to one liner, with no liner on the opposite surface (“open face”). Generally, the chamber is inerted (e.g., the oxygen-containing room air is replaced with an inert gas, e.g., nitrogen) while the samples are e-beam cured, particularly when open-face curing.

A wide range of E-beam doses are suitable to the crosslinked pressure sensitive adhesive layer of the articles of this disclosure. In some embodiments, the pressure sensitive adhesive layer is crosslinked with an E-beam dose of 0.5-4.0 MegaRads.

The crosslinked pressure sensitive adhesive layer may be of any suitable thickness, depending upon the desired use. In some embodiments, the thickness will be at least 10 micrometers, up to 2 millimeters, and in some embodiments the thickness will be at least 20 micrometers up to 1 millimeter thick. A wide range of intermediate thicknesses are also suitable, such as 25-500 micrometers, 200-400 micrometers, and the like.

A wide variety of methods of preparing the adhesive articles of this disclosure are suitable. Typically, the (meth)acrylate-based polymer is prepared by mixing the monomeric components and initiator to form a reaction mixture. In some embodiments, the components are dispersed in a solvent or mixture of solvents. Suitable solvents include hydrocarbon solvents such a hexane, heptane and the like, aromatic solvents such as benzene or toluene, or esters such as ethyl acetate. The reaction mixture is polymerized by activating the initiator (typically by heating above the activation temperature of the initiator). The desired additives (tackifier and plasticizer, if used) are then added to the polymerized mixture and the resultant mixture is coated onto a surface (typically a release liner), dried by heating, and cured. Curing involves exposure to E-beam radiation as described above and may also involve exposure to UV light if a photocrosslinker was incorporated into the (meth)acrylate-based polymer. If crosslinked on a release liner the resultant pressure sensitive adhesive layer can then be laminated to the surface of a desired substrate. A wide variety of release liners are suitable. Release liners are commonly used and well understood in the adhesive arts. Exemplary release liners include those prepared from paper (e.g., Kraft paper) or polymeric material (e.g., polyolefins such as polyethylene or polypropylene, ethylene vinyl acetate, polyurethanes, polyesters such as polyethylene terephthalate, and the like, and combinations thereof). At least some release liners are coated with a layer of a release agent such as a silicone-containing material or a fluorocarbon-containing material. Exemplary release liners include, but are not limited to, liners commercially available from CP Film (Martinsville, Va.) under the trade designation “T-30” and “T-10” that have a silicone release coating on polyethylene terephthalate film.

Also disclosed herein are medical constructions. In some embodiments, the medical constructions comprise a surface comprising mammalian skin, and an adhesive article adhesively attached to the surface. The adhesive article comprises the articles describe above. In some embodiments, the adhesive articles comprise a substrate with a first major surface and a second major surface, and a pressure sensitive adhesive layer disposed on at least a portion of the first major surface of the substrate. The pressure sensitive adhesive layer comprises an E-beam cured composition comprising a (meth)acrylate-based polymer that is free from acidic-functional or amide-functional groups, at least one tackifier comprising a hydrogenated hydrocarbon resin; and optionally contains at least one hydrocarbon-based plasticizer. The pressure sensitive adhesive layer has a static shear of at least 600 minutes to PROTEIN LEATHER.

As mentioned above, it is desirable that the adhesive article be removable from mammalian skin without causing skin damage. In some embodiments, the adhesive article is removable after 5 days. In many embodiments, the adhesive article is removable after longer periods of time such as 7 days, 14 days, 21 days, 30 days or longer. Typically, skin damage is determined by a physical inspection of the site to which the adhesive article was attached.

The components of the adhesive article, including suitable substrates and pressure sensitive adhesive layers are described in detail above.

### EXAMPLES

All percentages are by weight unless noted otherwise.

TABLE 1
Materials.

| | Acronym or | |
|---|---|---|
| Material | Designation | Source (Location) |
| 2-Ethylhexyl acrylate | 2EHA | Nippon Shokubai Co., Ltd. (Osaka, |
| | | Japan) |
| N-Vinylpyrrolidone | NVP | Nippon Shokubai Co., Ltd. (Osaka, |
| | | Japan) |
| Acryloyloxy Benzophenone, 25 wt % | ABP 25% in | 3M Company (St. Paul, MN) |
| in isooctyl acrylate | IOA | |
| 2-(2-cyanobutan-2-yldiazeny1)-2- | VAZO67 | FUJIFILM Wako Pure Chemical |
| methylbutanenitrile | | Corp. (Osaka, Japan) |
| Ethyl Acetate | EtOAC | FUJIFILM Wako Pure Chemical |
| | | Corp. (Osaka, Japan) |
| Toluene | Tol | FUJIFILM Wako Pure Chemical |
| | | Corp. (Osaka, Japan) |
| FILMBYNA TSB/TSC, Double side | TSC | FUJIMORI KOGYO Co., Ltd. |
| silicone-coated 50 μm PET film | | (Tokyo, Japan) |
| ARKON P-100, Hydrogenated | P-100 | Arakawa Chemical Industries, Ltd. |
| Hydrocarbon resin | | (Osaka, Japan) |
| SYLVARES Terpene Phenol | TP2019 | Kraton Corp. (Houston, TX) |
| Aliphatic C5 hydrocarbon resin | WINGTAC | Cray Valley USA LLC (Exton, PA) |
| | 95 | |
| Acrylic polymer solution, | PSA-1 | 3M Company (St. Paul, MN) |
| IOA/NVP = 91/9 | | |
| Acrylic polymer solution, | PSA-2 | 3M Company (St. Paul, MN) |
| IOA/AA = 98/2 | | |
| Iso-octyl Palmitate | IOP | Nikko Chemicals Co., Ltd. (Tokyo, |
| | | Japan) |
| PRIPLAST 3197, Polyester polyol | 3197 | Croda International Plc (Snaith, UK) |
| SONTARA 8010, PET spunlace | Sontara | Nissei. Ltd. (Nagano, Japan) |
| fabric | | |
| 3M Medical Tape 4076 | 4076 | 3M Company (St. Paul, MN) |
| PROTEIN LEATHER PBZ13001 | PROTEIN | IDEATEX Japan Co. Ltd. (Tokyo, |
| KAKI | LEATHER | Japan) |

### Study 1: Examples E1-E8 and Comparative Examples C1-C4

Monomers, initiator, and solvent were mixed in a glass jar according to the polymer formulations provided in Table 2. After nitrogen purging of the solution for 2 minutes (min), polymerization was carried out at 60° C. for 24 hour (hr), resulting in a viscous polymer solution.

The resulting polymer solutions were mixed with additives P-100 and/or 3197 according to the formulations provided in Table 3 and then coated on the surface of TSC using a knife coater. Additive P-100 acts as a tackifier, and 3197 acts as a plasticizer. The coated TSC was dried in an oven (70° C. for 2 min, followed by 120° C. for 2 min). Upon curing using either ultraviolet (UV) irradiation or an electron beam (E beam) as provided in Table 3, 100 micrometer (μm) thick pressure sensitive adhesive (PSA) sheets were obtained. Sontara was then laminated on the PSA sheets by 120° C. hot can lamination after corona treatment (corona treatment was conducted on the PSA surface with a corona generator AGF-B10 (Kasuga Denki Inc. at 0.15 kW). Hot can lamination is described in U.S. Pat. No. 6,703,108 (Bacon et al.).

TABLE 2
Study 1 polymer formulations.

| | 2EHA | NVP | VAZO67 | EtOAC | ABP 25% in IOA |
|---|---|---|---|---|---|
| Polymer 1 | 91 | 9 | 0.1 | 150 | |
| Polymer 2 | 91 | 9 | 0.1 | 150 | 0.16 |

TABLE 3
Study 1 PSA formulations.

| | Polymer | | | | | | |
|---|---|---|---|---|---|---|---|
| | solution | Components (g) | | | | E beam | UV |
| | (from Table | Polymer | | | | dose | dose |
| | 2) | Solution | P-100 | 3197 | Toluene | (MRad) | (mJ) |
| C1 | Polymer 1 | 75.76 | 0.00 | 0.00 | 24.24 | 2 | |
| C2 | Polymer 1 | 75.76 | 0.00 | 0.00 | 24.24 | 4 | |
| E1 | Polymer 1 | 68.18 | 3.03 | 0.00 | 28.79 | 2 | |
| E2 | Polymer 1 | 68.18 | 3.03 | 0.00 | 28.79 | 4 | |
| E3 | Polymer 1 | 60.61 | 6.06 | 0.00 | 33.33 | 2 | |
| E4 | Polymer 1 | 60.61 | 6.06 | 0.00 | 33.33 | 4 | |
| E5 | Polymer 1 | 64.39 | 3.03 | 1.52 | 31.82 | 2 | |
| E6 | Polymer 1 | 64.39 | 3.03 | 1.52 | 31.82 | 4 | |
| E7 | Polymer 1 | 60.61 | 6.06 | 0.00 | 33.33 | 2 | |
| E8 | Polymer 1 | 60.61 | 6.06 | 0.00 | 33.33 | 4 | |
| C3 | Polymer 2 | 60.61 | 6.06 | 0.00 | 33.33 | | 20 |
| C4 | Polymer 2 | 60.61 | 6.06 | 0.00 | 33.33 | | 40 |

The substrate was PROTEIN LEATHER PBZ13001 KAKI PROTEIN LEATHER, available from IDEATEX Japan Co. Ltd. (Tokyo, Japan)). PROTEIN LEATHER is a synthetic material having a surface of polyurethane blended with protein powder. The surface properties and elastic properties of PROTEIN LEATHER are such that we have found it to be a useful stretchable substrate to mimic testing on human skin.

The substrate was prepared by applying a coating of synthetic sebum. Synthetic sebum solution prepared according to Table 4 was coated on PROTEIN LEATHER using a D-bar coater (No. 30) and dried at 70° C., providing synthetic sebum-coated PROTEIN LEATHER. Blanks cut from the coated PROTEIN LEATHER were approximately 30 mm×125 mm.

TABLE 4
Synthetic sebum solution (31%).

| | Compounding (g) |
|---|---|
| Olive oil | 5.5 |
| Oleic acid | 2.5 |
| Squalene | 2 |
| Heptane | 12.24 |
| IPA | 9.36 |

### Static Shear Test

Sample tapes were prepared that were 25 mm×75 mm in size, and a 25 micrometers thick polyester (polyethylene terephthalate, PET) film was laminated on the upper 50 mm area of each sample tape to prevent stretch release of the sample tape.

A PET film laminated sample tape was laminated on one end of a coated PROTEIN LEATHER blank with 25 mm overwrapping, then they were compressed by application of a 2 kilogram (kg) roller (round trip, 50 millimeters per second (mm/sec)), provided a prepared test specimen.

Addition prepared test specimens were fixed in a 145-DP holding power tester (YASUDA SEIKI Company (Tokyo, Japan)). After the chamber conditions reached 40° C. and 75% relative humidity (RH), the prepared test specimens were held at these conditions for 4 hr without any weight loading. After four hours had elapsed, 300 g weights were applied to each specimen and the holding times were measured.

Static Peel Test with PROTEIN LEATHER

Sample tapes were prepared with dimensions 12.5 mm×125 mm, and a tab was created by folding one end of the sample tape. A tape sample was laminated on synthetic sebum-coated PROTEIN LEATHER, then compressed with 2 kg roller (round trip, 50 mm/sec).

One end of the laminated PROTEIN LEATHER was fixed to a jig, then a clip was attached to the tab of the laminated tape sample. A 100 g weight was attached to the clip, starting the static T peel test. The test time was 10 min, and the traveling length of delaminated tape was measured. If the tape was completely delaminated before 10 min, the estimated traveling time was calculated based on the fall off time according to ((laminating length)/(fall-off time)×(10 min)).

### Test Results

The results of static shear testing on synthetic sebum-coated PROTEIN LEATHER and static peel testing on synthetic sebum-coated PROTEIN LEATHER are provided in Table X. All trial samples showed good static shear performance at suitable curing levels (EB: 2 megarad (MRad), UV: 20 millijoule (mJ)) compared with commercial 3M Medical Tape 4076 (Comparative Example C5). Curing at higher levels resulted in the PSA losing flexibility and showing decreased stress relaxation performance, and the PSA delaminated from PROTEIN LEATHER There was no significant static shear performance difference in the trial formulations. The most important factor to achieve superior static shear performance is thought to be having a suitable polymer network that can provide both good cohesion and stress relaxation.

TABLE 5
Static Shear and Static Peel Test Results

| | Static Shear on PROTEIN | Static Peel on PROTEIN |
|---|---|---|
| Example | LEATHER (min) | LEATHER (min) |
| C1 | 3614 | 909 |
| C2 | 57 | — |
| E1 | 1752 | 52 |
| E2 | 195 | — |
| E3 | 3562 | 79 |
| E4 | 198 | — |
| E5 | 2479 | 205 |
| E6 | 189 | — |
| E7 | 836 | 222 |
| E8 | 462 | — |
| C3 | 3409 | 57 |
| C4 | 642 | — |
| C5 | 142 | 578 |

In the static peel test, it can be seen that PSA formulated with P-100 tackifier had improved performance. The tackifier provided good initial stickiness and resistance to peel force. Addition of 3197 plasticizer can improve wettability to skin, and at least in this trial, 5% plasticizer loading provided acceptable static peel performance.

Another factor is that to minimize medical adhesive-related skin injury (MARSI) risk, chemically stable PSA formulations are preferable. Residual monomer or photoinitiator may cause skin sensitivity. Although ABP can provide a good polymer network by suitable UV curing, there is risk that residual benzophenone moieties could irritate skin. Especially for long term wear, PSAs containing residual monomer or photoinitiator may not be preferable for skin application.

### Study 2: Examples E9-E12

Monomers, initiator, and solvent were mixed in a glass jar according to the formulations provided in Table 6. After nitrogen purging of the solution for 2 min, polymerization was carried out at 60° C. for 24 hour, and a viscous polymer solution was prepared.

The resulting polymer solutions were mixed with additives as provided in Table 7, coated on the surface of TSC using a knife coater, and then dried in an oven (70° C. for 2 min, followed by 120° C. for 2 min). After 3 MRad E beam curing, 100 μm thick PSA sheets were obtained. Sontara was then laminated on the cured PSA sheet by 120° C. hot can lamination after corona treatment as described above. Formulation and test condition detail is in Table 6 and 7.

TABLE 6
Study 2 polymer formulations.

| | 2EHA | NVP | VAZO67 | EtOAC |
|---|---|---|---|---|
| Polymer 3 | 85 | 15 | 0.1 | 150 |
| Polymer 1 | 91 | 9 | 0.1 | 150 |
| Polymer 4 | 95 | 5 | 0.1 | 150 |
| Polymer 5 | 98 | 2 | 0.1 | 150 |

TABLE 7
Study 2 PSA formulations.

| | | Polymer | Components (g) | | | | E beam |
|---|---|---|---|---|---|---|---|
| | De- | solution | Polymer | P- | | | dose |
| | scription | (from Table 6) | solution | 100 | IOP | Toluene | (MRad) |
| E9 | NVP-15 | Polymer 3 | 48.13 | 5 | 0.75 | 29.5 | 3 |
| E10 | NVP-9 | Polymer 1 | 48.13 | 5 | 0.75 | 29.5 | 3 |
| E11 | NVP-5 | Polymer 4 | 48.13 | 5 | 0.75 | 29.5 | 3 |
| E12 | NVP-2 | Polymer 5 | 48.13 | 5 | 0.75 | 29.5 | 3 |

Static Shear Test with Uncoated PROTEIN LEATHER

Test specimens for static shear testing were prepared as described in Study 1, except that the PROTEIN LEATHER was not coated with synthetic sebum.

Static shear testing was conducted as described in Study 1, except that the chamber conditions were 30° C. and 75% RH. The maximum test time was 1440 min. Results are provided in Table 8. These results confirmed the level of NVP in the polymer that was preferable for long term wear applications. More than 5% NVP loading may be needed to get adequate cohesion.

TABLE 8
Static Shear on Dry PROTEIN LEATHER

| | Static Shear on Dry PROTEIN LEATHER |
|---|---|
| Example | (min) |
| E9 | 1440 |
| E10 | 1440 |
| E11 | 42 |
| E12 | 5 |

### Study 3: Examples E13-E20

Polymers 1 and 6 were synthesized as provided in Table 9 by mixing the monomers, initiator, and solvent in a glass jar. After nitrogen purging of the solution for 2 min, polymerization was carried out at 60° C. for 24 hour, and a viscous polymer solution was prepared.

The resulting polymer solutions were compounded as provided in Table 10, coated on TSC surface by use of a knife coater, and then dried in oven (70° C. for 2 min, followed by 120° C. for 2 min). After E beam curing with each condition as provided in Table 8, 100 micrometer thick PSA sheets were obtained. Sontara was then laminated on the PSA sheet by 120° C. hot can lamination after corona treatment.

TABLE 9
Study 3 polymerization formulation.

| | 2EHA | NVP | VAZO67 | EtOAC |
|---|---|---|---|---|
| Polymer 1 | 91 | 9 | 0.1 | 150 |
| Polymer 6 | 91 | 9 | 0.1 | 230 |

TABLE 10
Study 3 Tape sample making.

| | | Components (g) | | | | E beam |
|---|---|---|---|---|---|---|
| | Polymer solution | Polymer | | | | dose |
| Example | (from Table 7) | solution | P-100 | IOP | Toluene | (MRad) |
| E13 | Polymer 1 | 48.13 | 5 | 0.75 | 29.5 | 0 |
| E14 | Polymer 1 | 48.13 | 5 | 0.75 | 29.5 | 1 |
| E15 | Polymer 1 | 48.13 | 5 | 0.75 | 29.5 | 3 |
| E16 | Polymer 1 | 48.13 | 5 | 0.75 | 29.5 | 6 |
| E17 | Polymer 6 | 64.17 | 5 | 0.75 | 13.5 | 0 |
| E18 | Polymer 6 | 64.17 | 5 | 0.75 | 13.5 | 1 |
| E19 | Polymer 6 | 64.17 | 5 | 0.75 | 13.5 | 3 |
| E20 | Polymer 6 | 64.17 | 5 | 0.75 | 13.5 | 6 |

Static Shear Test with Uncoated PROTEIN LEATHER

Static shear test specimens were prepared and as described above using uncoated Protein Leather, using chamber conditions of 30° C. and 75% RH. Results are provided in FIG. 5. In this study we confirmed effect of molecular weight of acrylic polymer. Polymer 1 has greater molecular weight than Polymer 6, and it showed better static shear performance. In the case of the low molecular weight polymer, higher crosslink levels were needed to achieve adequate cohesion. However, the polymer loses stress relaxation performance at high levels of crosslinking. Therefore, it is not easy to find a suitable level of crosslinking for a low molecular weight polymer. Use of a higher molecular weight polymer should be preferable for long term wear applications.

TABLE 11
Static Shear on Dry PROTEIN LEATHER

| | Static Shear on Dry PROTEIN LEATHER |
|---|---|
| Example | (min) |
| E13 | 0 |
| E14 | 0 |
| E15 | 1440 |
| E16 | 200 |
| E17 | 0 |
| E18 | 0 |
| E19 | 10 |
| E20 | 200 |

### Study 4: Examples E21-E50

Coatable PSA solutions were prepared according to the formulations provided in Table 12, coated on paper liner using a knife coater, and dried in an oven. After E beam curing with each condition, 100 micrometer thick PSA sheet were obtained. For static shear testing, Sontara was laminated on each PSA sheet by 120° C. hot can lamination after corona treatment. For tack testing, PET was laminated on the PSA sheet using a hand roller.

TABLE 12
Study 4 tape sample formulations.

| | Formulation (g) | | | | | |
|---|---|---|---|---|---|---|
| Example | PSA-1 | PSA-2 | TP2019 | Wingtac95 | P-100 | 3197 |
| E21 | 90 | | | | 10 | 0 |
| E22 | 85 | | | | 10 | 5 |
| E23 | 80 | | | | 10 | 10 |
| E24 | 80 | | | | 20 | 0 |
| E25 | 75 | | | | 20 | 5 |
| E26 | 70 | | | | 20 | 10 |
| E27 | 60 | | | | 20 | 20 |
| E28 | 50 | | | | 20 | 30 |
| E29 | 70 | | | | 30 | 0 |
| E30 | 90 | | 10 | | | |
| E31 | 80 | | 20 | | | |
| E32 | 70 | | 30 | | | |
| E33 | 90 | | | 10 | | |
| E34 | 80 | | | 20 | | |
| E35 | 70 | | | 30 | | |
| E36 | | 90 | | | 10 | 0 |
| E37 | | 85 | | | 10 | 5 |
| E38 | | 80 | | | 10 | 10 |
| E39 | | 80 | | | 20 | 0 |
| E40 | | 70 | | | 20 | 10 |
| E41 | | 60 | | | 20 | 20 |
| E42 | | 50 | | | 20 | 30 |
| E43 | | 70 | | | 30 | 0 |
| E44 | | 90 | 10 | | | 0 |
| E45 | | 85 | 10 | | | 5 |
| E46 | | 80 | 20 | | | 0 |
| E47 | | 70 | 30 | | | 0 |
| E48 | | 90 | | 10 | | 0 |
| E49 | | 80 | | 20 | | 0 |
| E50 | | 70 | | 30 | | 0 |

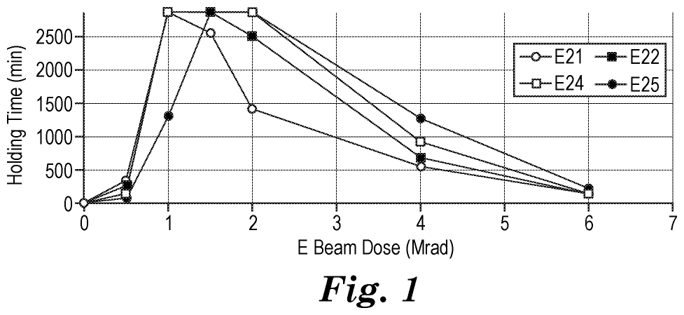

Static shear test specimens were fabricated as described above using synthetic sebum-coated PROTEIN LEATHER. A synthetic sebum solution was prepared according to Table 13 and coated on PROTEIN LEATHER using a D-bar coater (No. 30) followed by drying at 70° C., providing synthetic sebum-coated PROTEIN LEATHER. Static shear testing results are provided for Examples E21, E22, E24, and E25 at various levels of E-beam dose and shown in FIG. 1. FIG. 1 illustrates the impact of electron beam dose on PSA crosslink density. The results show that the PSA may have optimum properties at an intermediate level of crosslinking. Low levels of crosslinking resulted in cohesive failure, and high levels showed poor peel properties.

TABLE 13
Synthetic Sebum Solution (13%).

| | Formulation (g) |
|---|---|
| Olive oil | 5.5 |
| Oleic acid | 2.5 |
| Squalene | 2 |
| Heptane | 36.72 |
| IPA | 28.08 |

### Finger Tack Test

PSA tack stability was evaluated in a PSA open face situation. The PET-laminated sample was fixed on cardboard with the adhesive surface exposed to air. The fixed sample was maintained at ambient lab conditions for 3 months. The tackiness of the PSA was then checked using a finger tack test according to the criteria in Table 14.

TABLE 14
Finger tack evaluation.

| Score | Description |
|---|---|
| No | no tack |
| Low tack | some finger tack but not like day 1 |
| Yes | tack continues to be perfect, same as day 1 |

Open face stability of PSA tack is an important performance characteristic for long term applications. When the PSA is used to attach a medical device to the skin, the device/PSA combination may be stored exposed to air in an applicator without a liner. If there is any issue in compatibility of tackifier and acrylic polymer, tackifier migrates to the PSA surface and the PSA loses tackiness. Based on the result provided in Table 15, combination of PSA-1 and P-100 was only one solution in the trial.

TABLE 15
Result of Finger tack evaluation.

| | Formulation (g) | | | | | | Finger Tack |
|---|---|---|---|---|---|---|---|
| Ex- | PSA- | PSA- | | | P- | | (according to |
| ample | 1 | 2 | TP2019 | Wingtac95 | 100 | 3197 | Table 14) |
| E21 | 90 | | | | 10 | 0 | yes |
| E22 | 85 | | | | 10 | 5 | yes |
| E23 | 80 | | | | 10 | 10 | yes |
| E24 | 80 | | | | 20 | 0 | yes |
| E26 | 70 | | | | 20 | 10 | yes |
| E27 | 60 | | | | 20 | 20 | yes |
| E28 | 50 | | | | 20 | 30 | yes |
| E29 | 70 | | | | 30 | 0 | yes |
| E30 | 90 | | 10 | | | | no |
| E31 | 80 | | 20 | | | | no |
| E32 | 70 | | 30 | | | | no |
| E33 | 90 | | | 10 | | | low tack |
| E34 | 80 | | | 20 | | | low tack |
| E35 | 70 | | | 30 | | | low tack |
| E36 | | 90 | | | 10 | 0 | no |
| E37 | | 85 | | | 10 | 5 | no |
| E38 | | 80 | | | 10 | 10 | low tack |
| E39 | | 80 | | | 20 | 0 | no |
| E40 | | 70 | | | 20 | 10 | low tack |
| E41 | | 60 | | | 20 | 20 | yes |
| E42 | | 50 | | | 20 | 30 | yes |
| E43 | | 70 | | | 30 | 0 | no |
| E44 | | 90 | 10 | | | 0 | no |
| E45 | | 85 | 10 | | | 5 | no |
| E46 | | 80 | 20 | | | 0 | no |
| E47 | | 70 | 30 | | | 0 | no |
| E48 | | 90 | | 10 | | 0 | no |
| E49 | | 80 | | 20 | | 0 | no |
| E50 | | 70 | | 30 | | 0 | no |

1. A pressure sensitive adhesive article, comprising:
a substrate with a first major surface and a second major surface; and
a pressure sensitive adhesive layer disposed on at least a portion of the first major surface of the substrate, the pressure sensitive adhesive layer comprising an E-beam cured composition comprising:
a (meth)acrylate-based polymer that is free from acidic-functional or amide-functional groups; and
at least one tackifier comprising a hydrogenated hydrocarbon resin,
wherein the pressure sensitive adhesive layer has a static shear of at least 600 minutes to PROTEIN LEATHER.

2. The pressure sensitive adhesive article of claim 1, further comprising at least one hydrocarbon-based plasticizer.

3. The pressure sensitive adhesive article of claim 1, wherein the (meth)acrylate-based polymer is present in the pressure sensitive adhesive layer in an amount of at least 70% by weight.

4. The pressure sensitive adhesive article of claim 1, wherein the (meth)acrylate-based polymer is prepared from a polymerized reaction mixture, comprising:
at least 75 parts by weight of a first (meth)acrylate monomer of a formula:
CH₂═CR¹—(CO)—OR²
wherein R¹ is hydrogen or a methyl group; and
R² is an alkyl, heteroalkyl, or aryl group;
at least one co-polymerizable polar monomer that is free of acidic or amide functional groups; and
at least one free radical initiator.

5. The pressure sensitive adhesive article of claim 4, wherein the first (meth)acrylate monomer comprises an alkyl (meth)acrylate monomer with 4-12 carbon atoms.

6. The pressure sensitive adhesive article of claim 4, wherein the co-polymerizable polar monomer comprises NVP (N-vinyl pyrrolidone).

7. The pressure sensitive adhesive article of claim 4, wherein the reaction mixture further comprises at least one co-polymerizable monomer.

8. The pressure sensitive adhesive article of claim 1, wherein the pressure sensitive adhesive layer is crosslinked with an E-beam dose of 0.5-4.0 MegaRads.

9. The pressure sensitive adhesive article of claim 1, wherein the adhesive article is removable from mammalian skin after adhesion for at least 5 days without causing damage to the mammalian skin.

10. The pressure sensitive adhesive article of claim 1, wherein the substrate comprises at least one material selected from the group consisting of a polymeric film, a fabric, a non-woven, a foam, a paper, a mesh, and a release liner.

11. An adhesive article configured to attach to mammalian skin, the adhesive article comprising:
a substrate with a first major surface and a second major surface; and
a pressure sensitive adhesive layer disposed on at least a portion of the first major surface of the substrate, the pressure sensitive adhesive layer comprising an E-beam cured composition comprising:
a (meth)acrylate-based polymer that is free from acidic-functional or amide-functional groups;
at least one tackifier comprising a hydrogenated hydrocarbon resin; and
at least one hydrocarbon-based plasticizer,
wherein the pressure sensitive adhesive layer has a static shear of at least 600 minutes to PROTEIN LEATHER.

12. The adhesive article of claim 11, wherein the adhesive article is removable from the mammalian skin after at least 21 days without damaging the mammalian skin.

13. The adhesive article of claim 11, wherein the (meth)acrylate-based polymer is present in the pressure sensitive adhesive layer in an amount of at least 70% by weight.

14. The adhesive article of claim 11, wherein the (meth)acrylate-based polymer is prepared from a polymerized reaction mixture, comprising:
at least 75 parts by weight of a first (meth)acrylate monomer of a formula:
CH₂═CR¹—(CO)—OR²
wherein R¹ is hydrogen or a methyl group; and
R² is an alkyl, heteroalkyl, or aryl group;
at least one co-polymerizable polar monomer that is free of acidic or amide functional groups; and
at least one free radical initiator.

15. The adhesive article of claim 14, wherein the first (meth)acrylate monomer comprises an alkyl (meth)acrylate monomer with 4-12 carbon atoms.

16. The adhesive article of claim 14, wherein the co-polymerizable polar monomer comprises NVP (N-vinyl pyrrolidone).

17. The adhesive article of claim 14, wherein the reaction mixture further comprises at least one co-polymerizable monomer.

18. The adhesive article of claim 11, wherein the pressure sensitive adhesive layer is crosslinked with an E-beam dose of 0.5-4.0 MegaRads.

19. The adhesive article of claim 11, wherein the adhesive article is removable from the mammalian skin after adhesion for at least 5 days without causing damage to the mammalian skin.

20. The adhesive article of claim 11, wherein the substrate comprises at least one material selected from the group consisting of a polymeric film, a fabric, a non-woven, a foam, a paper, a mesh, and a release liner.
